(19) United States
(12) Patent Application Publication
(10) Pub. No.: US 2025/0213888 A1
(43) Pub. Date: Jul. 3, 2025

(54) RADIOTHERAPY SUPPORT APPARATUS, RADIOTHERAPY APPARATUS, AND RADIOTHERAPY SUPPORT METHOD

(71) Applicant: Hitachi High-Tech Corporation, Tokyo (JP)

(72) Inventors: Takao KIDANI, Tokyo (JP); Masato SUZUKI, Tokyo (JP); Shin YANAGISAWA, Tokyo (JP)

(21) Appl. No.: 18/850,602

(22) PCT Filed: Feb. 15, 2023
(86) PCT No.: PCT/JP2023/005293

§ 371 Date: Sep. 25, 2024

(30) Foreign Application Priority Data
May 31, 2022 (JP) 2022-089163

(51) Int. Cl. A61N 5/10

(52) U.S. Cl. CPC A61N 5/1049; A61N 2005/1061

(57) ABSTRACT

Provided is a radiotherapy support apparatus capable of reducing the burden on an operator and a patient in radiotherapy. An input unit 42 receives patient information indicating at least one of an irradiation site of a patient A irradiated with radiation and physical characteristics of the patient A. A workflow generation unit 46 generates, based on the patient information, workflow information that is setting information indicating setting contents for performing radiotherapy in a radiotherapy apparatus 2 that irradiates the patient A with radiation.

### TECHNICAL FIELD

The present disclosure relates to a radiotherapy support apparatus, a radiotherapy apparatus, and a radiotherapy support method.

### BACKGROUND ART

In radiotherapy in which a patient is irradiated with radiation to treat an affected area such as a tumor, it is necessary for an operator to manually set item values for various items according to patient information such as a patient's body shape and treatment site. For example, the operator needs to select a positioning method for performing positioning of moving a patient lying on a couch to a position suitable for treatment according to patient information, and then set each item corresponding to the positioning method one by one.

In addition, the setting items are different according to the patient information, and there are various setting items such as imaging conditions for capturing an image necessary for positioning and a type of additional work performed by the operator on the patient. For this reason, there are many operations of the operator, and a burden is imposed on the operator. Furthermore, this becomes a factor that prolongs the treatment time, and the burden on the patient also increases.

On the other hand, PTL 1 discloses a technology for notifying information regarding a next procedure according to a progress status of a series of procedures at the time of imaging based on workflow information defining the content and order of work performed in the series of procedures related to imaging for acquiring a medical image in a medical image diagnostic apparatus such as a magnetic resonance imaging (MRI) apparatus. According to this technology, it is possible to present a procedure related to imaging, and thus, it is possible to improve the work efficiency of the operator.

### CITATION LIST

### Patent Literature

PTL 1: JP 2018-183525 A

### SUMMARY OF INVENTION

### Technical Problem

In radiotherapy, unlike imaging in a medical image diagnostic apparatus such as the technology described in PTL 1, a positioning method with a different imaging method and setting information such as setting items specific to radiotherapy other than imaging and item values thereof are different according to patient information. Therefore, the technology described in PTL 1 cannot prepare efficient workflow information in radiotherapy in advance. For this reason, the technology described in PTL 1 has a problem that the burden on the operator and the patient cannot be reduced in the radiotherapy.

An object of the present disclosure is to provide a radiotherapy support apparatus, a radiotherapy apparatus, and a radiotherapy support method capable of reducing the burden on an operator and a patient in radiotherapy.

### Solution to Problem

A radiotherapy support apparatus according to one aspect of the present disclosure is a radiotherapy support apparatus for supporting radiotherapy performed by a radiotherapy apparatus that irradiates a patient with radiation, the radiotherapy support apparatus including: an input unit that receives patient information indicating at least one of an irradiation site of the patient irradiated with radiation and physical characteristics of the patient; and a generation unit that generates, based on the patient information, setting information indicating a plurality of setting items and an item value that is a value of each setting item as setting contents to be set for performing the radiotherapy.

### Advantageous Effects of Invention

According to the present invention, it is possible to reduce a burden on an operator and a patient in radiotherapy.

### BRIEF DESCRIPTION OF DRAWINGS

FIG. 1 is a diagram illustrating a radiotherapy system according to a first embodiment of the present disclosure.

FIG. 2 is a diagram illustrating an example of template data management information.

FIG. 3 is a view illustrating an example of a setting screen.

FIG. 4 is a flowchart for describing an operation of a workflow management system according to the first embodiment of the present disclosure.

FIG. 5 is a diagram illustrating an example of workflow information.

FIG. 6 is a flowchart for describing an operation of a workflow management system according to a second embodiment of the present disclosure.

### DESCRIPTION OF EMBODIMENTS

Hereinafter, embodiments of the present disclosure will be described with reference to the drawings.

### First Embodiment

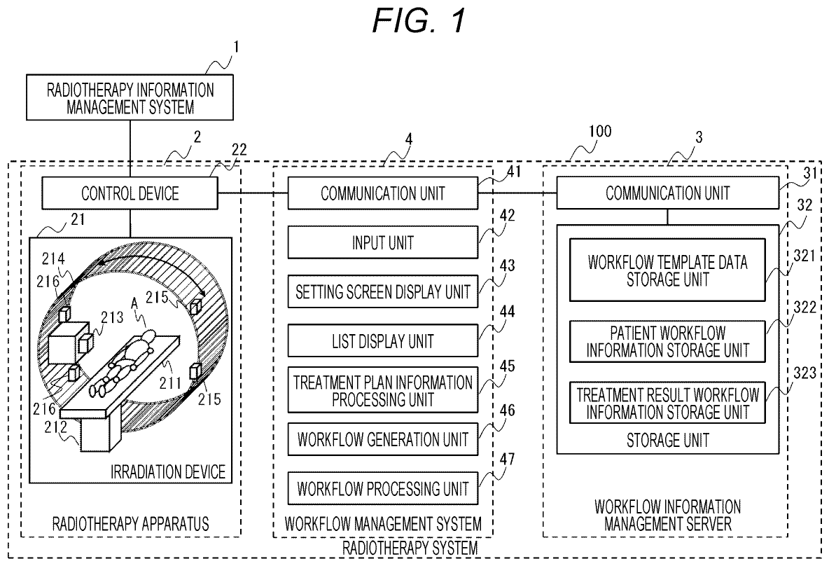

FIG. 1 is a diagram illustrating a radiotherapy system according to a first embodiment of the present disclosure. A radiotherapy system 100 illustrated in FIG. 1 includes a radiotherapy apparatus 2, a workflow information management server 3, and a workflow management system 4. The radiotherapy system 100 is communicatively connected to the radiotherapy information management system 1.

The radiotherapy information management system 1 manages treatment plan information and treatment result information received from a treatment planning system (not illustrated). The radiotherapy information management system 1 transmits treatment plan information to the radiotherapy apparatus 2. The radiotherapy is usually performed in a plurality of times. In the present embodiment, the radiotherapy is performed on a plurality of treatment days. The treatment plan information indicates, for example, irradiation contents such as an irradiation area, an irradiation energy, an irradiation angle, and the number of times of irradiation with radiation with which a patient A is irradiated as a treatment plan of radiotherapy for the patient A.

Based on treatment plan information from the radiotherapy information management system 1 and workflow information from the workflow management system 4 to be described below, the radiotherapy apparatus 2 performs positioning of the patient A by an image acquired in the treatment room and emits radiation. The radiotherapy apparatus 2 includes an irradiation device 21 and a control device 22.

The irradiation device 21 is a device that irradiates the patient A with radiation. The irradiation device 21 includes a couch 211, a moving device 212, an irradiation nozzle 213, a gantry 214, a radiation generator 215, and a radiation detector 216. The couch 211 is a table on which the patient A is placed. The moving device 212 moves the couch 211. The irradiation nozzle 213 irradiates the patient A placed on the couch 211 with radiation. For example, the irradiation nozzle 213 irradiates the patient A with radiation transported from an accelerator (not illustrated) via a transport system (not illustrated). The gantry 214 adjusts the irradiation angle of the radiation from the irradiation nozzle 213 with respect to the patient A by rotating the irradiation nozzle 213 around the patient. The type of radiation applied to the patient A is not particularly limited, and may be a particle beam or an electromagnetic wave such as an X-ray. The radiation generator 215 and the radiation detector 216 constitute a radiation imaging device that acquires a radiographic image for performing positioning processing of the patient A to be described below. The radiation generator 215 generates radiation such as X-rays, and the radiation detector 216 detects the radiation. There are a plurality of sets of the radiation generator 215 and the radiation detector 216, and the radiation generator 215 and the radiation detector 216 of each set are arranged so as to pinch the irradiation port of the irradiation nozzle 213 and so that the paths of the respective radiations are orthogonal to each other. Although two sets of the radiation generator 215 and the radiation detector 216 are illustrated in the drawing, three or more sets may be provided.

The control device 22 controls the irradiation device 21 based on treatment plan information, workflow information, various operations from an operator who is a user of the radiotherapy system 100, and the like, and causes the irradiation device 21 to irradiate the patient A with radiation. For example, the control device 22 controls the moving device 212 to execute a positioning processing or the like for moving the affected area of the patient A placed on the couch 211 to a position suitable for radiation irradiation. More specifically, the positioning processing is a process of bringing the affected area of the patient A into the same position state as at the time of creating the treatment plan using a CT image obtained by imaging the patient A in advance in order to create the treatment plan or a DRR image which is a digitally reconstructed X-ray image generated from the CT image, and the radiographic image captured by the radiation imaging device including the radiation generator 215 and the radiation detector 216.

The workflow information management server 3 and the workflow management system 4 are radiotherapy support apparatuses that support radiotherapy performed by the radiotherapy apparatus 2, and generate workflow information that is setting information indicating setting contents for performing radiotherapy in the radiotherapy apparatus 2. The workflow information includes, for example, a plurality of setting items and an item value that is a value of each setting item as setting contents. In addition, the workflow information may further indicate the order and timing of performing the work related to each setting item.

The workflow information management server 3 is a management device that manages workflow information and information for generating the workflow information, and includes a communication unit 31 and a storage unit 32.

The communication unit 31 communicates with the workflow management system 4 to input and output information. The storage unit 32 stores various types of information regarding the workflow information. Specifically, the storage unit 32 includes a workflow template data storage unit 321, a patient workflow information storage unit 322, and a treatment result workflow information storage unit 323.

The workflow template data storage unit 321 stores a plurality of pieces of template data that are candidates for the workflow information and template data management information that is management information for managing the template data.

The template data includes, for example, as setting items, a positioning method which is a processing method of positioning processing executed by the radiotherapy apparatus 2, a positioning-related item determined in relation to the positioning method, an algorithm of the positioning processing, an implementation method related to re-planning of a treatment plan, a setup couch position which is an initial position of the couch 211 in a first radiotherapy, additional medical work which is additional work performed by a medical worker in relation to radiotherapy, necessity of confirmation of misalignment of the patient A before and after radiotherapy, and the like. The implementation method related to the re-planning of the treatment plan may include a verification method of the re-planned treatment plan.

Examples of the setting value of the positioning method include a 2D3D method, a 3D3D method, and a 2D2D method. The 2D3D method is a method in which positioning is performed using a 3D image which is a three-dimensional radiographic image such as a computed tomography (CT) image at the time of planning a treatment plan and using a 2D image which is a two-dimensional radiographic image such as an X-ray image is performed on a treatment day on which radiotherapy is performed. The 3D3D method is a method using 3D images both at the time of creation and on the day of treatment, and the 2D2D method is a method using 2D images both at the time of creation and on the day of treatment.

The positioning-related content includes, for example, at least one of a gradation of a radiographic image (2D image and 3D image) used in a positioning processing, an imaging condition related to imaging for acquiring a radiographic image, and whether or not adaptive treatment (adaptive radiotherapy) is performed. The adaptive treatment is a treatment method in which a treatment plan is re-planned in a case where it is assumed that a dose cannot be appropriately administered according to the treatment plan due to changes in the body shape, organ position, and the like of the patient A within the treatment period. In the case of determining the necessity of adaptive treatment, a 3D3D positioning method using a 3D image on the day of treatment is empirically more appropriate. The imaging condition may include an imaging angle (gantry angle) at which the 2D image is acquired and an imaging angle range for acquiring the 3D image of the cone beam CT.

Specifically, the algorithm of the positioning processing is an algorithm for automatically performing positioning based on the radiographic image. The implementation method is, for example, implementation timing of performing necessity determination processing for determining necessity of re-planning of a treatment plan in adaptive radiotherapy. The necessity determination processing includes CT imaging or the like for determining the necessity of re-planning of the treatment plan. The implementation timing is defined by, for example, the number of times of irradiation.

The additional medical work is, for example, insertion work of inserting a balloon catheter into the patient A, anesthesia treatment of administering anesthesia to the patient A, and the like.

Note that the above-described setting items are merely examples, and the setting items are not limited thereto. The setting items can include any items to be set for radiotherapy and work to be performed related to radiotherapy.

Specifically, the template data management information is information for managing a correspondence relationship between patient information related to a patient and template data applied to the patient. In the present embodiment, the patient information includes an irradiation site, which is a site where radiotherapy of the patient is performed (a site where radiation is irradiated), and physical characteristics of the patient. The physical characteristics may, for example, indicate classifications such as small, standard and large. In addition to these classifications, the physical characteristics may indicate a physically immature child. In addition, the physical characteristics may be measured values regarding the body such as weight, height, and chest circumference, calculated values calculated from the measured values, and the like. In addition, the patient information does not need to include both the irradiation site and the physical characteristics, and may include at least one of the irradiation site and the physical characteristics.

Each setting item of the template data may not be determined according to all of the patient information. For example, among the setting items of the template data, the algorithm of the positioning processing, the implementation method related to the re-planning of the treatment plan, the verification method of the re-planned treatment plan, the setup couch position, and the necessity of confirmation of the misalignment of the patient A before and after the radiotherapy treatment may be determined according to the irradiation site.

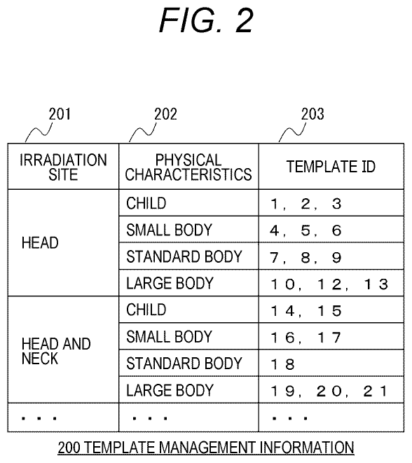

FIG. 2 is a diagram illustrating an example of template data management information. Template data management information 200 illustrated in FIG. 2 includes fields 201 to 203.

The fields 201 and 202 store patient information. Specifically, the field 201 stores an irradiation site of the patient, and field the 202 stores physical characteristics of the patient. The field 203 stores a template data ID for specifying a template data candidate. The template data management information may store other information such as a name of each pieces of template data.

The description returns to FIG. 1. The patient workflow information storage unit 322 stores the workflow information for each patient.

The treatment result workflow information storage unit 323 stores treatment result workflow information which is workflow information generated based on treatment results of radiotherapy. Note that the treatment result workflow information will be described in more detail in the second embodiment.

The workflow management system 4 is a radiotherapy support apparatus that generates workflow information for the patient A. The workflow management system 4 includes a communication unit 41, an input unit 42, a setting screen display unit 43, a list display unit 44, a treatment plan information processing unit 45, a workflow generation unit 46, and a workflow processing unit 47.

The communication unit 41 communicates with the radiotherapy apparatus 2 and the workflow information management server 3 to input and output information.

The input unit 42 receives various types of information such as patient information from an operator who is a user of the radiotherapy system 100. Furthermore, the input unit 42 may receive various types of information from other devices such as the radiotherapy apparatus 2 and the workflow information management server 3 via the communication unit 41.

The setting screen display unit 43 generates and displays a setting screen that is a screen for setting patient information of the patient A to be subjected to radiotherapy.

The list display unit 44 generates and displays a template data list indicating each piece of template data corresponding to the patient information of the patient A received by the input unit 42 as a candidate for the workflow information of the patient A based on the template data management information stored in the workflow template data storage unit 321. For example, the list display unit 44 displays the template data list in addition to the setting screen.

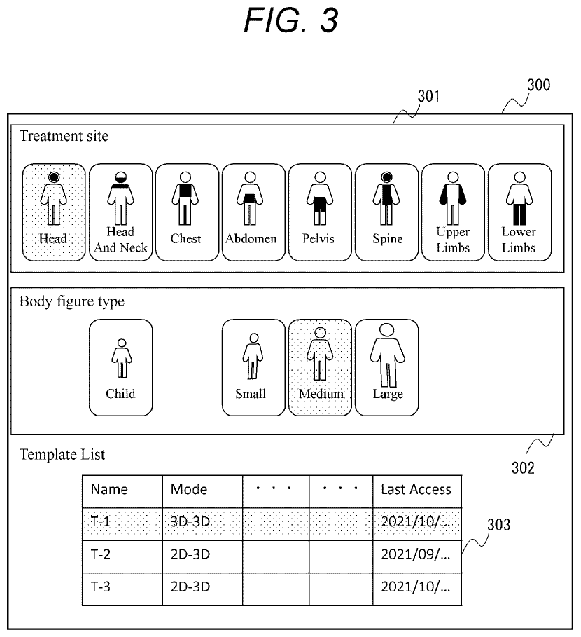

FIG. 3 is a diagram illustrating an example of a setting screen including a template data list. The setting screen 300 illustrated in FIG. 3 includes an irradiation site selection unit 301 that selects an irradiation site, a body shape selection unit 302 that selects physical characteristics, and a list display area 303 that displays a template data list. The list display area 303 may also display information on each piece of template data together. In the example of FIG. 3, the type of positioning method corresponding to the template data list and the like are displayed. Note that, in the example of FIG. 3, the physical characteristics are classified into four of child, small body, standard d body, and large body, but the present invention is not limited to this example.

The description returns to FIG. 1. The treatment plan information processing unit 45 acquires treatment plan information from the radiotherapy information management system 1 or the radiotherapy apparatus 2 via the communication unit 41.

The workflow generation unit 46 is a generation unit that generates workflow information for the patient A based on the patient information of the patient A received by the input unit 42. In the present embodiment, the workflow generation unit 46 generates, as workflow information, selected workflow data that is template data selected from a template data list corresponding to patient information.

At this time, the workflow generation unit 46 may determine at least a part of the item value of each item of the workflow information according to the treatment plan information. For example, the workflow generation unit 46 may determine an imaging angle (gantry angle) at which a 2D image is acquired, an imaging angle range for acquiring a 3D image of the cone beam CT, and the like based on a plurality of gantry angles at which radiation is emitted in the treatment plan information.

The workflow processing unit 47 displays the workflow information generated by the workflow generation unit 46 and edits the workflow information in accordance with an instruction from the operator. Then, the workflow processing unit 47 stores the edited workflow information in the patient workflow information storage unit 322 of the workflow information management server 3.

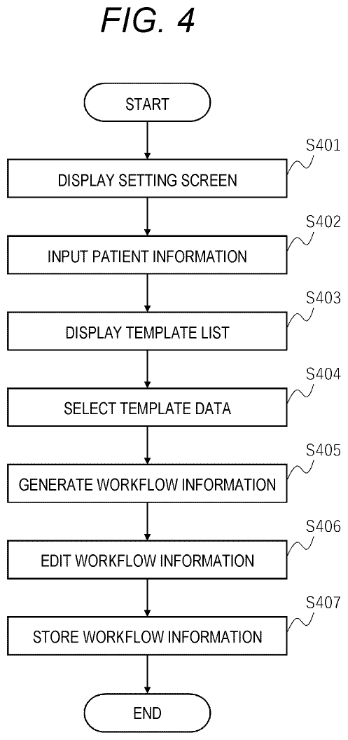

FIG. 4 is a flowchart for describing an operation of the workflow management system 4. The following operation is performed, for example, before the first radiotherapy is performed.

First, the setting screen display unit 43 of the workflow management system 4 displays a setting screen (see FIG. 3) for setting patient information (step S401).

Thereafter, the input unit 42 receives patient information selected by the operator using the setting screen (step S402). Then, the list display unit 44 confirms the template data management information stored in the workflow template data storage unit 321 of the workflow information management server 3 via the communication unit 41. The list display unit 44 generates a template data list indicating each piece of template data corresponding to the patient information received by the input unit 42 as a candidate for the workflow information of the patient A based on the template data management information, and adds the template data list to the setting screen (step S403).

Then, the input unit 42 receives selection information indicating the selected template data selected by the operator among the template data included in the template list (step S404). The workflow generation unit 46 acquires the selected template data indicated by the selection information from the workflow template data storage unit 321 of the workflow information management server 3 based on the selection information, via the communication unit 41. The workflow generation unit 46 generates and displays the workflow information of the patient A based on the selected template data (step S405).

For example, the workflow generation unit 46 may set the selected template data as the workflow information of the patient A without change, or may set at least a part of the item values included in the selected template data based on the treatment plan information of the patient A as the workflow information. Note that the treatment plan information can be acquired using the treatment plan information processing unit 45.

Thereafter, the workflow processing unit 47 edits the workflow information generated by the workflow generation unit 46 (step S406). For example, the workflow processing unit 47 first displays the workflow information. Then, when the input unit 42 receives an editing instruction of the workflow information from the operator, the workflow processing unit 47 edits the workflow information based on the editing instruction. The editing of the workflow information is, for example, change of an item value of an item included in the workflow information, addition of an item, deletion of an item, or the like. Furthermore, in a case where it is not necessary to edit the workflow information, the processing of step S406 is skipped.

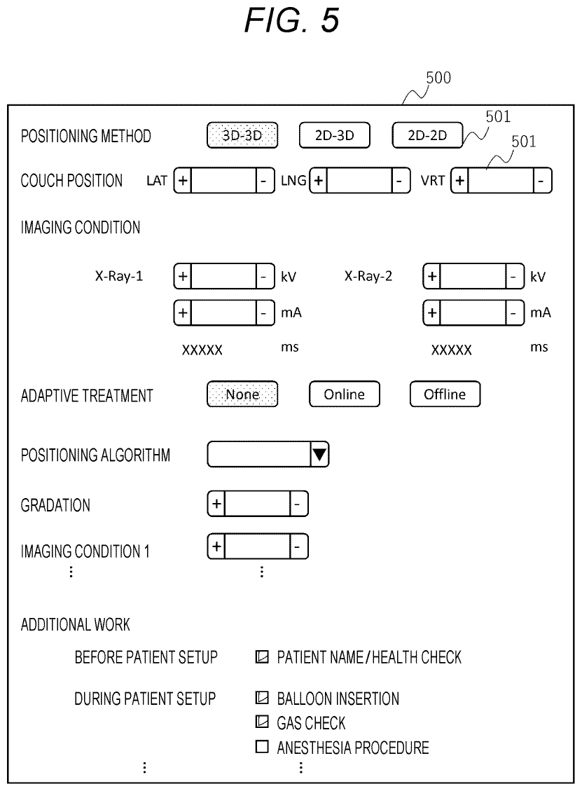

FIG. 5 is a diagram illustrating an example of the workflow information displayed in step S406. The workflow information 500 illustrated in FIG. 5 includes an interface 501 for displaying and editing the item value for each setting item. The interface 501 is, for example, a button, a drop-down list, a check box, or the like. Note that the setting items illustrated in FIG. 5 are merely examples, and differ depending on patient information.

The description returns to FIG. 4. The workflow processing unit 47 stores the edited workflow information in the patient workflow information storage unit 322 of the workflow information management server 3 via the communication unit 41 (step S407), and ends the processing.

Note that the stored workflow information is displayed on the radiotherapy apparatus 2, the workflow management system 4, or the like before and after radiotherapy and during radiotherapy, for example, on each treatment day of radiotherapy. As a result, the operator can perform radiotherapy while confirming the displayed workflow information. In addition, the control device 22 of the radiotherapy apparatus 2 may automatically perform various settings for performing radiotherapy based on the workflow management information.

In addition, the workflow generation unit 46 may generate the workflow information without using the template data. In this case, for example, management information indicating a correspondence relationship between each value of the patient information and a combination thereof and each item and item value of the workflow information may be stored instead of the template data management information, and the workflow generation unit 46 may generate the workflow information based on the management information.

As described above, according to the present embodiment, the input unit 42 receives patient information indicating the irradiation site of the patient A irradiated with radiation and the physical characteristics of the patient A. The workflow generation unit 46 generates, based on the patient information, workflow information that is setting information indicating setting contents for performing radiotherapy in the radiotherapy apparatus 2 that irradiates the patient A with radiation. Therefore, since it is possible to generate the workflow information according to the patient information, it is possible to reduce the burden on the operator and the patient in the radiotherapy.

Furthermore, in the present embodiment, the workflow generation unit 46 generates the workflow information indicating the positioning method and the positioning-related content determined according to the positioning method based on the patient information. Therefore, it is possible to appropriately generate the workflow information regarding the positioning processing.

In the present embodiment, the positioning-related content includes the gradation of the radiographic image used in the positioning processing, the imaging condition related to imaging for acquiring the radiographic image, and necessity of adaptive radiotherapy. The radiographic image is, for example, an X-ray image, and the imaging condition includes an X-ray diaphragm position for acquiring the X-ray image, an imaging angle (gantry angle) for acquiring a 2D image, and an imaging angle range for acquiring a 3D image of cone beam CT. Therefore, it is possible to more appropriately generate the workflow information regarding the positioning processing.

Furthermore, in the present embodiment, the workflow generation unit 46 generates workflow information indicating an algorithm of positioning processing. Therefore, it is possible to appropriately generate the workflow information regarding the algorithm of the positioning processing.

Furthermore, in the present embodiment, the workflow generation unit 46 generates workflow information regarding re-planning of a treatment plan regarding radiotherapy. Therefore, it is possible to appropriately generate the workflow information regarding the re-planning of the treatment plan.

In the present embodiment, the workflow generation unit 46 generates the workflow information indicating the setup couch position, which is the initial position of the couch 211 in the first radiotherapy. Therefore, it is possible to appropriately generate the workflow information regarding the setup couch position.

Furthermore, in the present embodiment, the workflow generation unit 46 generates workflow information indicating the additional medical work performed by the medical worker in relation to radiotherapy. Therefore, it is possible to appropriately generate the workflow information regarding the additional medical work.

Furthermore, in the present embodiment, the workflow generation unit 46 generates the workflow information for generating the setting information indicating the necessity of confirmation of the misalignment of the patient A before and after the radiotherapy treatment. Therefore, it is possible to appropriately generate the workflow information regarding the necessity of the image comparison after the radiotherapy.

Furthermore, in the present embodiment, the workflow generation unit 46 generates the workflow information based on the selection template selected according to the patient information. Therefore, appropriate workflow information can be easily generated.

### Second Embodiment

The configuration of the radiotherapy system of the present embodiment is similar to that of the radiotherapy system of the first embodiment illustrated in FIG. 1.

In the present embodiment, recommendation information recommended as workflow information is given in advance to any of template data corresponding to patient information.

In addition, the operator causes the radiotherapy apparatus 2 to perform the first radiotherapy without selecting template data from the template list. The control device 22 of the irradiation device 21 generates treatment result workflow information indicating a treatment result performed in the first radiotherapy, and transmits the treatment result workflow information to the communication unit 41 of the workflow management system 4.

The workflow generation unit 46 generates the template data to which the recommendation information is given as first workflow information that is workflow information corresponding to the first radiotherapy. Then, when the communication unit 41 receives the treatment result workflow information, the workflow generation unit 46 stores the treatment workflow information in the treatment result workflow information storage unit 323 of the workflow information management server 3, edits the first workflow information based on the treatment result workflow information, and generates the edited first workflow information as workflow information corresponding to the second and subsequent radiotherapies.

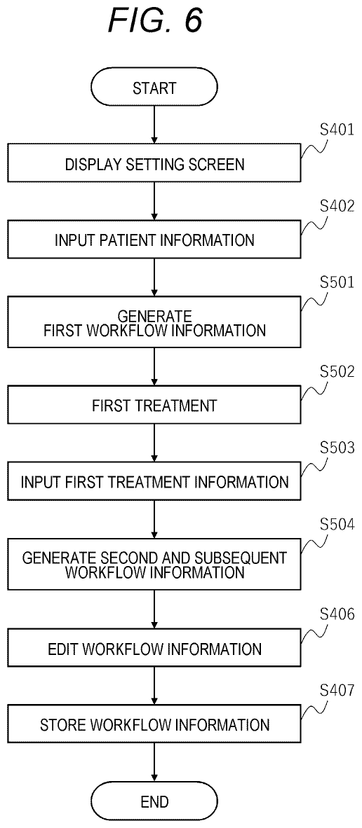

FIG. 6 is a flowchart for describing an operation of the workflow management system 4 of the present embodiment.

First, similarly to the operation of the first embodiment described with reference to FIG. 4, the processing of steps S401 and S402 is executed. Thereafter, the workflow generation unit 46 confirms the template data management information stored in the workflow template data storage unit 321 of the workflow information management server 3 via the communication unit 41. Based on the template data management information, the list display unit 44 generates and displays the template data to which the recommendation information is given among the template data corresponding to the patient information received by the input unit 42 as the first workflow information (step S501).

Thereafter, while confirming the displayed first workflow information, the operator performs first radiotherapy while performing setting (step S502). At this time, the operator may perform setting different from the setting content indicated by the first workflow information.

When the first radiotherapy is completed, the communication unit 41 receives the treatment result workflow information. The workflow generation unit 46 stores the treatment result workflow information in the treatment result workflow information storage unit 323 of the workflow information management server 3 (step S503).

The workflow generation unit 46 edits the first workflow information based on the treatment result workflow information and generates the edited first workflow information as the workflow information of the second and subsequent radiotherapies (step S504). For example, the workflow generation unit 46 reflects the difference between the first workflow information and the treatment result information in the first workflow information, thereby generating the difference as the workflow information of the second and subsequent radiotherapies.

Thereafter, the processing of steps S406 and S407 is executed, and the processing ends. The workflow information edited and stored in steps S406 and S407 may be only the workflow information of the second and subsequent radiotherapies.

In the present embodiment, the workflow information of the second and subsequent radiotherapies is generated based on the treatment result workflow information of the first radiotherapy. However, it is sufficient that the workflow information of the subsequent radiotherapy is generated based on the treatment result workflow information of the past radiotherapy. For example, the workflow information of the subsequent radiotherapy may be generated based on the latest (previous) treatment result workflow information of radiotherapy. In a case where the treatment result workflow information of the first radiotherapy is used, it is possible to more appropriately support the second and subsequent radiotherapies while reducing the burden on the operator and the patient in the first radiotherapy. On the other hand, in a where the latest treatment result workflow information of the radiotherapy is used, it is not necessary to manually edit the setting information for the item optimally corrected at the time of treatment, and it is possible to more appropriately support subsequent radiotherapy.

More preferably, the radiotherapy support apparatus according to one aspect of the present disclosure may be configured to generate setting information indicating, as setting contents, a positioning method that is a method of positioning processing in which the generation unit moves an affected area of a patient on a couch to a position for radiotherapy in the radiotherapy apparatus based on patient information, and positioning-related contents determined according to the positioning method.

More preferably, the radiotherapy support apparatus according to one aspect of the present disclosure may have a configuration in which positioning-related contents include at least one of gradation of a radiographic image used for positioning processing, an imaging condition related to imaging for acquiring a radiographic image, and necessity of adaptive radiotherapy.

More preferably, the radiotherapy support apparatus according to one aspect of the present disclosure may have a configuration in which the radiographic image is an X-ray image, and the imaging condition includes an X-ray diaphragm position for acquiring the X-ray image.

More preferably, in the radiotherapy support apparatus according to one aspect of the present disclosure, the generation unit may generate the setting information indicating the algorithm of the positioning processing as the setting content based on the irradiation site.

More preferably, in the radiotherapy support apparatus according to one aspect of the present disclosure, the generation unit may generate setting information including an implementation method regarding re-planning of a treatment plan regarding radiotherapy based on the irradiation site.

More preferably, in the radiotherapy support apparatus according to one aspect of the present disclosure, the generation unit may generate setting information indicating the initial position of the couch in the first radiotherapy as setting contents based on the irradiation site.

More preferably, in the radiotherapy support apparatus according to one aspect of the present disclosure, the generation unit may generate setting information indicating, as setting contents, additional medical work to be performed by a medical worker in relation to radiotherapy based on patient information.

More preferably, in the radiotherapy support apparatus according to one aspect of the present disclosure, the generation unit may generate setting information indicating necessity of confirmation of misalignment of the patient before and after radiotherapy treatment as setting contents based on the irradiation site.

More preferably, the radiotherapy support apparatus according to one aspect of the present disclosure may further include a display unit that displays a plurality of pieces of template data corresponding to received patient information based on management information for managing a correspondence relationship between patient information and the plurality of pieces of template data that are candidates for setting information, and the generation unit may be configured to generate template data selected from the plurality of pieces of displayed template data as the setting information.

More preferably, in the radiotherapy support apparatus according to one aspect of the present disclosure, the radiotherapy may be performed in a plurality of times, the input unit may receive treatment result information indicating a treatment result which is setting contents set when the radiotherapy was performed in the past radiotherapy, and the generation unit may generate past setting information which is setting information corresponding to the past radiotherapy based on the patient information, and may edit the past setting information based on the treatment result information to generate setting information corresponding to the subsequent radiotherapy.

A radiotherapy apparatus according to one aspect of the present disclosure includes a radiotherapy support apparatus for supporting radiotherapy performed by a radiotherapy apparatus that irradiates a patient with radiation, and may include: an input unit that receives patient information indicating at least one of an irradiation site to which radiation is applied in the patient and physical characteristics of the patient; a generation unit that generates, based on the patient information, setting information indicating a plurality of setting items and an item value that is a value of each setting item as setting contents for performing radiotherapy; and a control device that performs setting for performing radiotherapy based on the setting information generated by the generation unit.

A radiotherapy support method according to one aspect of the present disclosure is a radiotherapy support method using a radiotherapy support apparatus for supporting radiotherapy performed by a radiotherapy apparatus that irradiates a patient with radiation, in which patient information indicating at least one of an irradiation site to be irradiated with radiation in the patient and physical characteristics of the patient may be received, and setting information indicating a plurality of setting items and an item value that is a value of each setting item may be generated as setting contents for performing radiotherapy based on the patient information.

The above-described embodiments of the present disclosure are examples for describing the present disclosure, and are not intended to limit the scope of the present disclosure only to those embodiments. Those skilled in the art can practice the present disclosure in various other aspects without departing from the scope of the present disclosure. For example, the system configuration of the radiotherapy system 100 is not limited to the example illustrated in FIG. 1, and the functions of the workflow information management server 3 and the workflow management system 4 may be realized by one device, or the radiotherapy apparatus 2 may have at least one function of the workflow information management server 3 and the workflow management system 4.

### REFERENCE SIGNS LIST

1 radiotherapy information management system
2 radiotherapy apparatus
3 workflow information management server
4 workflow management system
21 irradiation device
22 control device
31 communication unit
32 storage unit
41 communication unit
42 input unit
43 setting screen display unit
44 list display unit
45 treatment plan information processing unit
46 workflow generation unit
47 workflow processing unit
100 radiotherapy system

1. A radiotherapy support apparatus for supporting radiotherapy performed by a radiotherapy apparatus that irradiates a patient with radiation, the radiotherapy support apparatus comprising:
an input unit that receives patient information including at least one of an irradiation site of the patient irradiated with radiation and physical characteristics of the patient; and
a generation unit that generates, based on the patient information, setting information indicating a plurality of setting items and an item value that is a value of each setting item as setting contents to be set for performing the radiotherapy.

2. The radiotherapy support apparatus according to claim 1, wherein the generation unit generates, based on the patient information, the setting information indicating a positioning method that is a method of positioning processing of moving an affected area of the patient on a couch to a position for the radiotherapy in the radiotherapy apparatus and positioning-related contents determined according to the positioning method as the setting contents.

3. The radiotherapy support apparatus according to claim 2, wherein the positioning-related contents include at least one of a gradation of a radiographic image used in the positioning processing, an imaging condition regarding imaging for acquiring the radiographic image, and necessity of adaptive radiotherapy.

4. The radiotherapy support apparatus according to claim 3, wherein
the radiographic image is an X-ray image, and
the imaging condition includes an X-ray diaphragm position for acquiring the X-ray image.

5. The radiotherapy support apparatus according to claim 2, wherein the generation unit generates, based on the irradiation site, the setting information indicating an algorithm of the positioning processing as the setting contents.

6. The radiotherapy support apparatus according to claim 1, wherein the generation unit generates, based on the irradiation site, the setting information including an implementation method regarding re-planning of a treatment plan regarding the radiotherapy.

7. The radiotherapy support apparatus according to claim 2, wherein the generation unit generates, based on the irradiation site, the setting information indicating an initial position of the couch in the first radiotherapy as the setting contents.

8. The radiotherapy support apparatus according to claim 1, wherein the generation unit generates, based on the patient information, the setting information indicating additional medical work to be performed by a medical worker in relation to the radiotherapy as the setting contents.

9. The radiotherapy support apparatus according to claim 1, wherein the generation unit generates, based on the irradiation site, the setting information indicating necessity of confirmation of misalignment of the patient before and after treatment of the radiotherapy as the setting contents.

10. The radiotherapy support apparatus according to claim 1, further comprising
a display unit that displays a plurality of pieces of template data corresponding to the received patient information based on management information for managing a correspondence relationship between the patient information and the plurality of pieces of template data that are candidates for the setting information,
wherein the generation unit generates template data selected from the plurality of pieces of displayed template data as the setting information.

11. The radiotherapy support apparatus according to claim 1, wherein
the radiotherapy is performed a plurality of times,
the input unit receives treatment result information indicating a treatment result which is setting contents set when the radiotherapy was performed in the past, and
the generation unit generates past setting information that is the setting information corresponding to the past radiotherapy based on the patient information, and edits the past setting information based on the treatment result information to generate the setting information corresponding to the subsequent radiotherapy.

12. A radiotherapy apparatus comprising a control device that performs setting for performing radiotherapy based on setting information generated by the radiotherapy support apparatus according to claim 1.

13. A radiotherapy support method using a radiotherapy support apparatus for supporting radiotherapy performed by a radiotherapy apparatus that irradiates a patient with radiation, the radiotherapy support method comprising:
receiving patient information indicating at least one of an irradiation site of the patient irradiated with radiation and physical characteristics of the patient; and
generating, based on the patient information, setting information indicating a plurality of setting items and an item value that is a value of each setting item as setting contents for performing the radiotherapy.
